(19) United States
(12) Patent Application Publication
(10) Pub. No.: US 2021/0205583 A1
(43) Pub. Date: Jul. 8, 2021

(54) CATHETERIZATION APPARATUS, CATHETER, AND METHOD

(71) Applicant: EndoWays LTD., Caesarea (IL)

(72) Inventor: Noam Shaul SHAMAY, Moshav Elyakhin (IL)

(73) Assignee: EndoWays LTD., Caesarea (IL)

(21) Appl. No.: 17/207,416

(22) Filed: Mar. 19, 2021

Related U.S. Application Data
(63) Continuation of application No. PCT/IL2019/051044, filed on Sep. 22, 2019.
(60) Provisional application No. 62/765,936, filed on Sep. 24, 2018.

(51) Int. Cl. A61M 25/01; A61M 25/00; G16H 20/40

(52) U.S. Cl. CPC A61M 25/0133; A61M 25/0108; A61M 2025/09083; G16H 20/40; A61M 2025/0042; A61M 25/0053

(57) ABSTRACT

A catheterization apparatus includes a catheter having a steering mechanism for deflecting a distal portion of the catheter by operation of relative bending stiffnesses of a drive tube and of a core wire. The catheter is remotely controlled from a control station via a rotatable actuation device which supports actuators for providing translation and rotation motions. The catheter is looped and rigidly guided in a channel controlling a distal length of the catheter.

### CROSS-REFERENCE TO RELATED APPLICATIONS

This application is a Continuation of International Application No. PCT/IL2019/051044, filed Sep. 22, 2019, which claims the priority of U.S. Provisional Patent Application No. 62/765,936, filed Sep. 24, 2018, the entire contends of both of which are incorporated herein by reference.

### FIELD OF THE INVENTION

The embodiments described hereinbelow pertain to the fields of catheters, and in particular to steering mechanisms and translation mechanisms for the navigation of catheters in bodies.

### BACKGROUND OF THE INVENTION

A problem with existing technology is how to navigate an instrument or a probe through the tortuous and sinuous sharp angled branching of the bodily vessels of a human or an animal body. The bodily vessels may include for example those of the blood system, digestive system, urinary tracts, brain vasculature, respiratory, and other systems. Bodily vessels may branch-off at sharp angles making it difficult and often impossible to penetrate and navigate a catheter therethrough.

For the sake of illustration, one may consider a common catheter having a wire GW with a distal end which is bent into a curved distal J-shaped hook J, and is pushed from a proximal PRX to a distal DST direction, into a bodily vessel or conduit VSL, as shown in FIG. 1. FIG. 1 shows the disposition of the guide wire GW in the interior of a rather linear portion of the vessel VSL having walls WL shown as dashed lines. In such a disposition, the J-shaped hook J of the guide wire GW is easily pushed in distal progression. FIG. 1 also shows a bifurcation BFR forming an acute angle α with the bodily vessel or conduit VSL into a second vessel 2VSL. When the guide wire GW is pushed distally up to the bifurcation BFR until the curved J-shaped hook J abuts a corner of the bifurcation BFR, which becomes a support, the guide wire GW will easily engage the second vessel 2VSL.

However, as shown in FIG. 2, the problem is how to navigate a guide wire GW pushed in the distal direction DST in the vessel 3VSL, via the bifurcation 2BRF, which forms an obtuse angle β relative to the vessel 4VSL, and into the vessel 4VSL. Steering a guide wire GW from the vessel 3VSL and into the vessel 4VSL is a strenuous problem for a practitioner, and is almost impossible to achieve.

It would therefore be advantageous to provide a mechanism to facilitate the task and shorten the span of time spent by a practitioner when trying to navigate through sinuous bifurcations and to pass through tortuous vessels.

In some cases, the navigation problem becomes even harder when the targeted blood vessels are deep, thus far away distally, and require passage through several bifurcations. In such cases, the operation of the catheter becomes challenging and the need to deliver a push force through a long guide wire GW adds difficulty to the navigation problem.

The background art describes methods and apparatus configured to navigate a tube to a desired distal location within a lumen using guide wires having a pre-shaped distal portion. Other methods include control of the orientation of guide wires and catheters as they progress distally, but lack details about how the distally driven implement is pushed and/or rotated from a proximal end. There is thus a problem with long torturous vessels since the transmission of proximally delivered thrust force and radial rotation becomes difficult and less controllable.

Background documents pertaining to the field or the art include: U.S. Pat. No. 6,270,496, US 2008/015625 A1, US 2012/004504 A1, CN 108339188 A, CN 107753107 A, EP 2508120 A1, and US 2017/105605 A1.

### SUMMARY

It is an object of the embodiments of the present invention is to provide a catheterization apparatus including a catheter for navigation through body vessels. The catheter includes a resilient core wire deformed distally into a core wire bend to form a core wire nose which ends in a distal core wire tip. The catheter further includes a drive tube having a drive tube lumen holding the core wire therein. The drive tube is configured to operate in one out of two configurations. One configuration is a navigation configuration for navigation in bodily vessels wherein the core wire bend is supported in a straightened disposition in the drive tube lumen. Another configuration is a penetration configuration for entering a bifurcated vessel. Thereby, the core wire nose is configured to deflect a distal portion of the drive tube into a drive tube deflected arm.

Another object of the embodiments of the present invention is to provide a method for implementing a catheterization apparatus by providing a core wire distally deformed into a core wire bend, and providing a drive tube having a drive tube lumen holding the deformed core wire therein. Thereby, translating one of the core wire and the drive tube relatively to each other will dispose a steering mechanism in navigation mode or in penetration mode.

Still, another object of the embodiments of the present invention is to provide a flexible drive tube DT with an exterior surface supporting helically wound recessed microgrooves forming female screw threads adapted to receive therein tissue from the lumen. Thereby, rotation of the drive tube into protruding male screw threads formed by in the tissue received in the recessed microgrooves, drives the drive tube into translation.

It is yet another object of the embodiments of the present invention to provide a catheter wherein at least one of the drive tube and the core wire is configured to support a plurality of portions of length having different bending stiffness values. Thereby, relative mutual translation of the drive tube and the core wire commands a reversible deformation of shape of one of the drive tube and the core wire.

It is another object of the embodiments of the present invention to provide a method wherein the drive tube and the core wire have a plurality of portions of length having a bending stiffness of different value, which plurality of portions of length operate in relative mutual translation to command a controlled reversible deformation of shape of at least one of the drive tube and the core wire.

Yet still another object of the embodiments of the present invention is to provide a catheter including a drive tube which supports a core wire therein and an actuation device having a rotatable disk which is configured to provide mechanical support and motion to the catheter. Thereby, actuation orders, delivered by a handheld manually operated control station which is coupled in communication with the actuation device, controls translation and rotation of the drive tube and of the core wire.

One other object of the embodiments of the present invention is to provide a method providing a channel for mechanically confining and supporting a distal portion of the distal portion of the catheter therein. Further, providing rotation to the drive tube to enhance distal translation thereof into a target vessel, and arresting motion of the core wire relative to the target vessel while the turntable drives the drive tube into the target vessel.

Relative to the commonly available apparatus, a navigation catheter operating a controllable steering mechanism STMC including an orientable and lengthenable distal end portion which provides a user with superior capabilities for navigation into the sinuous ramifications of bodily vessels VSL. More advantages of the embodiments described hereinbelow will become apparent in the following description.

### BRIEF DESCRIPTION OF DRAWINGS

Non-limiting embodiments of the invention will be described with reference to the following description of exemplary embodiments, in conjunction with the figures. The figures are generally not shown to scale and any measurements are only meant to be exemplary and not necessarily limiting. In the figures, identical structures, elements, or parts that appear in more than one figure are preferably labeled with a same or similar number in all the figures in which they appear, in which:

FIGS. 1 to 4 illustrate the problem and the solution,

FIGS. 5 and 6 schematically illustrate an exemplary embodiment of the distal portion of the catheter CAT which has a steering mechanism STMC including a drive tube DT,

FIGS. 7 and 8 depict the erection process of the drive tube DT into a deflected arm,

FIG. 10 illustrates the catheter in straight navigation configuration,

FIGS. 11 to 13 relate to dispositions of the drive tube in a vessel,

FIGS. 14 to 16 illustrate a drive tube lumen formed as a stranded tube of coils,

FIGS. 17 to 19 detail control of the catheter for penetration in a branching vessel,

FIG. 20 is a lock diagram of the apparatus APP,

FIGS. 21 to 24 illustrate the principles of relative bending stiffness,

FIGS. 25 to 28 exemplify the use of relative bending stiffness,

FIGS. 29 and 30 refer to a plurality of bending stiffness portions of length,

FIGS. 31 to 35 exemplify penetration in aortic type III arch bifurcations,

FIG. 36 is a block diagram of the apparatus APP showing an actuation device,

FIGS. 37 and 38 illustrate an actuation device,

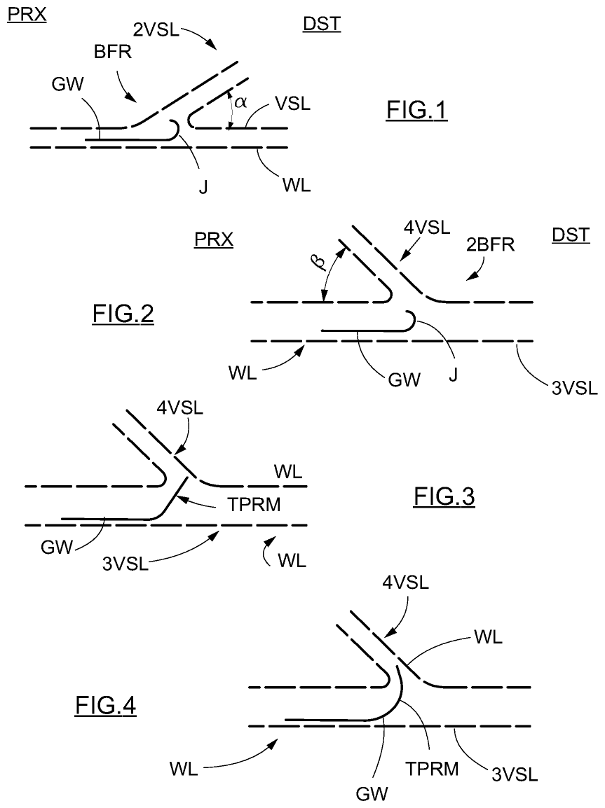

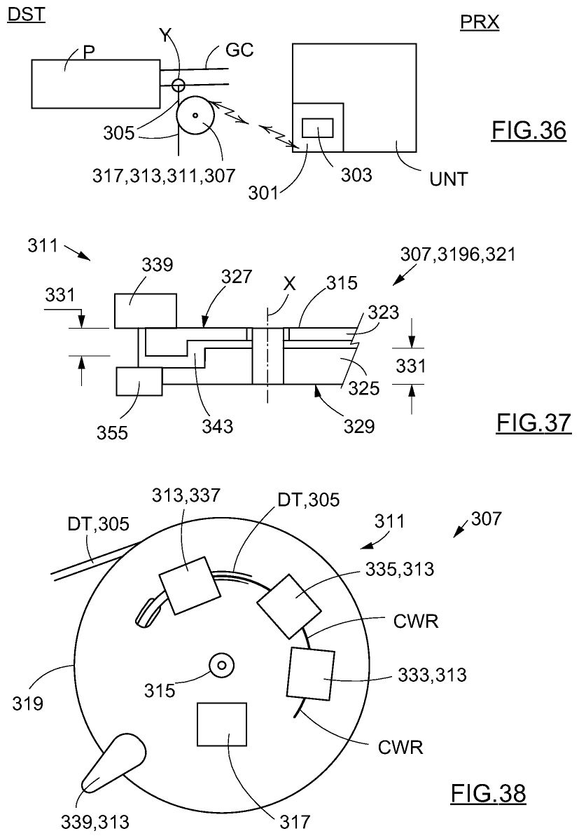

FIG. 36 is a top view of a control station,

FIGS. 40a through 40e depict examples of channel cross sections, and

FIG. 41 illustrates the loop of a drive tube in the rotatable turntable.

### DETAILED DESCRIPTION

One example of a catheter for navigation including a steering mechanism described in detail hereinbelow allows control of the distal extension and proximal retraction of the tip end of the navigation apparatus towards and away from a target location in a bodily vessel. The catheter includes a steering mechanism STMC which includes a deflected tip arm TPRM capable of radial orientation and having a controllable length.

FIG. 3 illustrates the tip arm TPRM as may be controllably oriented, lengthened and shortened by commands provided by a user located proximally, thus ex vivo, or otherwise generated automatically by an algorithm embedded in a control device which is optionally located in whole or in part within the body and optionally located in whole or in part on the exterior of the body. It is understood that the navigation process may be continuously visualized in real time by use of appropriate imaging facilities, and by radiopaque markers that may be located along the tip arm TPRM. FIG. 4 depicts a solution for the translation of the tip arm TPRM into the bifurcated vessel 4VSL, as described hereinbelow.

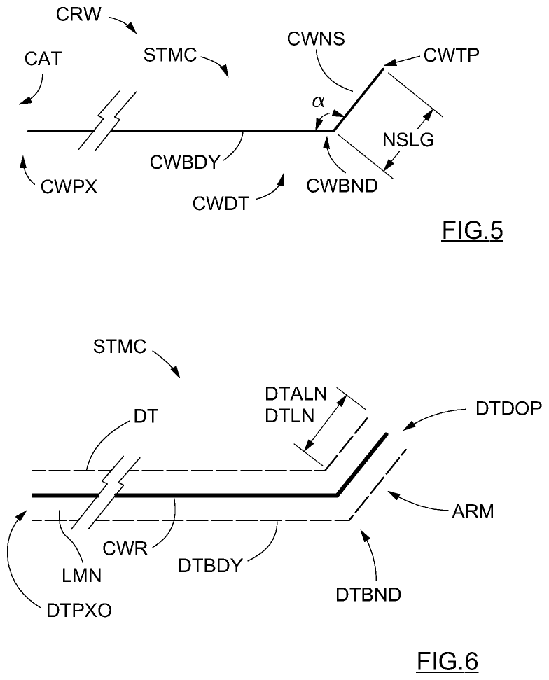

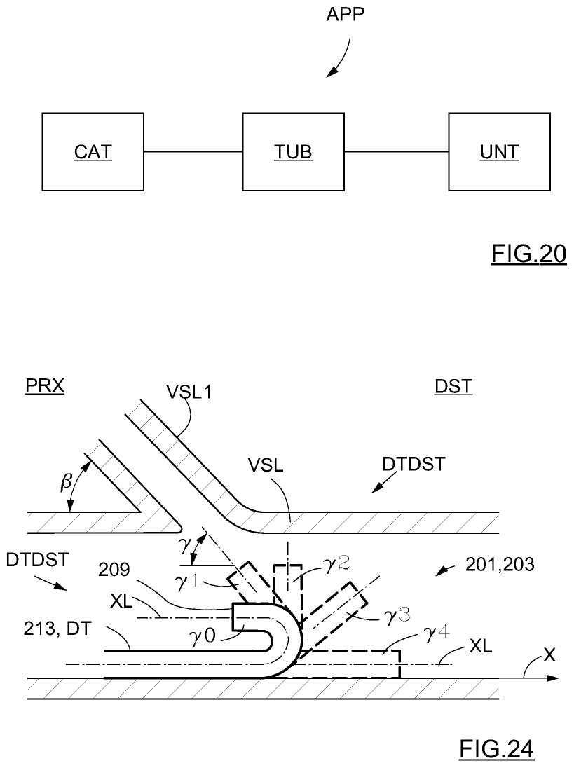

FIGS. 5 and 6 schematically illustrate an exemplary embodiment of the distal steering portion of the catheter CAT which has a steering mechanism STMC including a drive tube DT. The drive tube DT is a flexible tube having a lumen LMN which supports a core wire CRW therein. In some of the figures the drive tube DT is shown in dashed lines and the core wire CRW is shown as a single line. The steering mechanism STMC is disposed at the distal portion of a catheter CAT (FIG. 20). In this description, distal, distally, and distal direction, and synonyms thereof are referred to as DST. Proximal, proximally, and proximal direction, and synonyms thereof are referred to as PRX.

In FIG. 5, the core wire CRW, which is flexible and resilient, includes a core wire proximal portion CWPX, a core wire body portion CWBDY, and a core wire distal portion CWDT. The distal portion of the core wire distal portion CWDT is bent a priori in deformation at a core wire bend CWBND to form a core wire nose CWNS. The core wire bend CWBND, which is the transition portion between the core wire body portion CWBDY and the core wire nose CWNS, may form a desired angle α between the core wire body portion CWBDY and the core wire nose CWNS. The angle α may be acute or obtuse and the core wire bend CWBND may be rounded off. The portion of the core wire CRW extending distally away from the core wire bend CWBND forms a core wire nose portion CWNS which is terminated distally by a core wire tip CWTP. The core wire nose portion CWNS which extends from the core wire bend CWBND to the core wire tip CWTP may be a straight portion of core wire CWR which may have a fixed selected predetermined length indicated as nose length NSLG.

In FIG. 6, the drive tube DT is shown to have a drive tube proximal opening DTPXO, a drive tube distal opening DTDOP, and a drive tube bend DTBND conforming with the core wire bend CWBND. The drive tube proximal opening DTPXO and the drive tube distal opening DTDOP delimit the drive tube lumen DTLMN.

The drive tube lumen DTLMN of the drive tube DT holds the core wire CRW therein in freedom of motion in translation and in rotation. This holds true even when the drive tube DT has a drive tube bend and the core wire CRW has a core wire bend CWBND with a core wire nose CWNS, that are both confined in the interior of the drive tube lumen DTLMN.

Still in FIG. 6, the drive tube DT is shown in a disposition wherein the drive tube distal opening DTDOP is in flush disposition relative to the core wire nose tip NSTP. Since the core wire bend CWBND is more rigid than the drive tube DT, the drive tube DT conforms to the direction of orientation of the core wire nose CWNS. Hence, the drive tube DT bends to form a drive tube deflected arm ARM having a flush disposition length indicated as the distal tube distal portion flush disposition arm length DTLN.

FIG. 7 depicts the disposition of the drive tube DT after a first step of translation relative to the core wire CRW, whereby a proximal operation has driven the distal tube distal opening DTDOP distally away from the nose tip NSTP of the core wire CRW. Although being less rigid than the core wire CWR, the drive tube distal portion DTDST does extend along and maintains the direction of orientation of the core wire nose CWNS. The drive tube distal open opening DTDOP passed well away from the core wire nose tip CWTP, and in continuation of the orientation of the core wire nose CWNS. Thereby the distal portion of the drive tube DT of FIG. 7 has grown and forms a longer drive tube deflected arm DTARM, having a first step drive tube length DTLN1 which is longer than the flush disposition length DTLN depicted in FIG. 6.

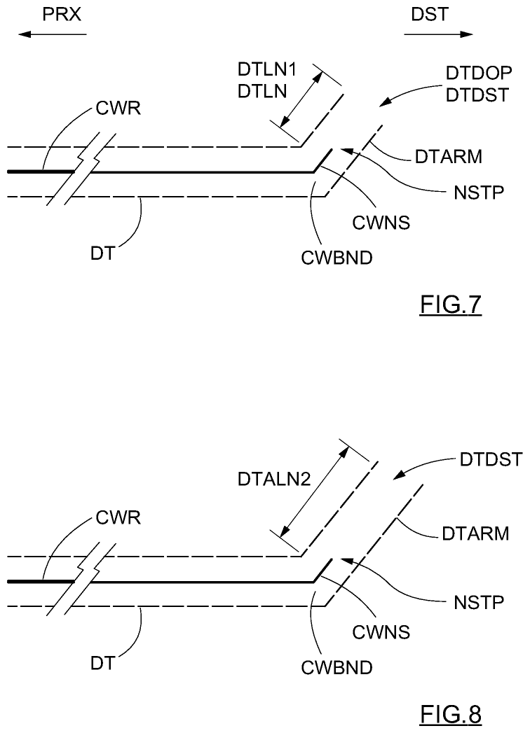

In FIG. 8, following a second step of translation of the drive tube DT relative to the core wire CRW, thus over the core wire nose CWNS and away from the nose tip NSTP, the length DTLN of the drive tube deflected arm DTARM has grown and has reached a length of DTLN2 which is longer than the length DTLN1 of the first step of translation.

Likewise, proximal translation of the drive tube DT may shorten the length DTLN of the drive tube deflected arm DTARM so that the drive tube distal opening DTDOP may return for example, to become disposed flush with the core wire tip CWTP, with a drive tube deflected arm DTARM of length DTALN, as shown in FIG. 6. This means that the length of the drive tube deflected arm DTARM is controllable. In other words, the displacement of either one of the drive tube DT and the core wire CRW relative to each other, controls the length of extension of the drive tube arm length DTLN. Hence, displacing the drive tube DT relative the core wire CRW or displacing the core wire CRW relative to the drive tube DT produces the same result and determines the length of extension of the drive tube arm length DTLN.

The core wire CRW is rotatable, whereby when rotated, the core wire nose CWNS will drive the drive tube deflected arm DTARM to rotate in accordance therewith. This means that the rotation of the core wire CRW allows rotation of the drive tube deflected arm DTARM which is thus controllably orientable in n times 360° of orientation, where n is a positive or negative real number. This means that the arm DTARM is controllably rotatable in radial orientation towards a bifurcation BFR for penetration into an open opening of a branching vessel VSL. This feature of controllable rotatable and radial orientation, in combination with the controllable relative mutual disposition of the core wire CRW within the drive tube DT allows for an accurate control of both the angle as well as the radial movement of the drive tube DT. It is clear from the figures and the above description that differently from the mode of action of many existing guide wires and micro catheter systems, in the proposed embodiment the core wire CRW does not need to extend beyond the distal opening of the drive tube. Note that the control of radius and orientation by a pre-shaped guide wire alone necessitate pre-selection of the point of bending which is hard to achieve when a variety of bifurcation at various angles need to be traveled because a different bending point of the wire will be typically required.

There has thus been described a steering mechanism STMC for a catheter CAT allowing to erect a drive tube deflected arm DTARM of controllable length DTLN at a predetermined angle α. The angle α may be acute or obtuse, and furthermore, the drive tube deflected arm DTARM may be directed in a radial orientation which may cover n×360°, where n is an integer.

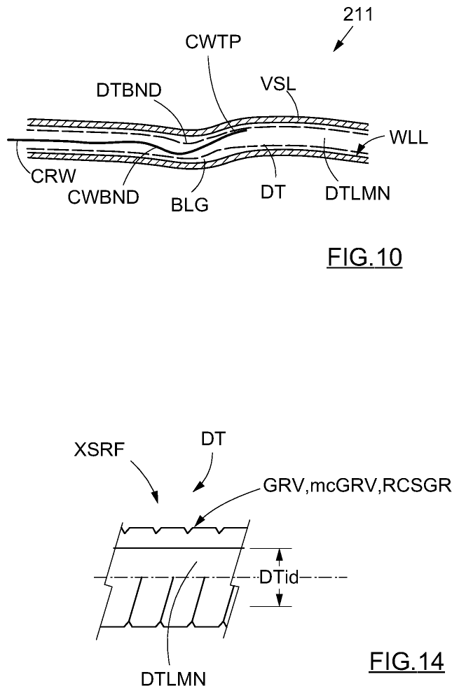

FIG. 10 schematically illustrates the catheter in straight navigation configuration. In this configuration, the CRW is not engaged with the distal portion of the DT and thus allows the operator to advance the catheter straight along the vessel VSL and to avoid entering a bifurcation BFR or changing the catheter path.

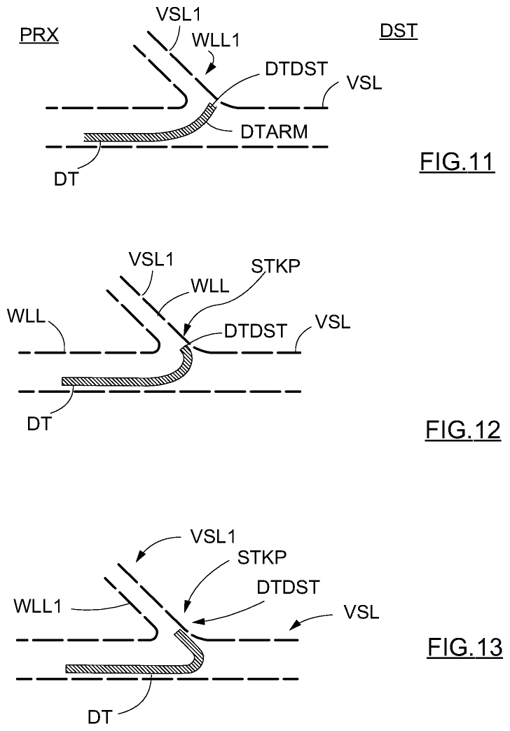

FIG. 11 depicts the drive tube deflected arm DTARM which has been navigated to enter a bifurcated vessel VSL1, the drive tube distal end DTDST of which has found support on a wall WLL1 of the vessel VSL1. The drive tube DT being flexible and of low rigidity, even small forces such as friction forces for example, may prevent further progress of the drive tube distal end DTDST into the vessel VSL1. Therefore, even though it is sometimes possible to use proximally applied push forces to urge the drive tube DT into the vessel VSL1, success is precarious and most often the navigation efforts fail.

FIG. 12 depicts the drive tube DT, the distal end DTDST of which has just entered in the interior of the vessel VSL1. A proximally delivered push force applied on the drive tube DT has not been able to further push the distal end DTDST to progress into the bifurcated vessel VSL1 which distal end DTDST has been stuck in place at point STKP, at the entrance of the vessel VSL1. Furthermore, in response to the proximally applied push force, a portion of the distal tube proximal to the distal end DTDST has started to buckle into the vessel VSL.

In FIG. 13, in response to additional proximally applied push forces, the distal portion of the drive tube DT is shown to have buckled further into the vessel VSL while the distal end DTDST is still stuck at point STKP in the bifurcation of vessel VSL1. Instead of progressing into the bifurcated vessel VSL1, the drive tube DT penetrated even further into the main vessel VSL. Proximally applied push forces delivered on the drive tube DT are thus of no avail.

To solve the problem caused by the drive tube DT getting stuck and buckling, advantage is taken from the inherent self-pull translation feature which is achieved by the rotation of the drive tube, as described in relation to FIG. 14.

FIG. 14 illustrates a detail of a flexible drive tube lumen DTLMN. The flexible drive tube DT has an exterior surface XSRF supporting a plurality or recessed grooves GRV formed therein as microgrooves mcGRV which, when in contact with an interior wall WLL of a vessel VSL, may receive therein tissue of that wall WLL. The recessed grooves GRV function like a recessed or female screw thread operative in association with vessel's wall tissue TSS that is received therein (FIG. 16). The translation of the flexible drive tube DT in response to the rotation thereof, is different from the translation of a bolt that is rotated in a nut. Contrary to a bolt, the drive tube DT has female, here recessed grooves GRV, wherein the tissue TSS of the interior walls WLL of the lumen of a vessel VSL penetrates and forms a kind of male protrusions. Hence, contrary to a male thread which traumatically penetrates into the tissue TSS of a lumen LMN of a vessel VSL, that same tissue TSS flows atraumatically into the smooth exterior microgrooves mcGRV of the drive tube DT.

For practical reasons of economy, the drive tube DT shown in FIG. 14 may preferably be acquired as a custom-made stranded coil-tube known for example under the name of Helical Hollow Strand or HHS, which are trademarks.

A stranded coil tube HHS is a flexible tube formed out of a plurality of prestressed helically coiled threads wound together and forming an interior lumen. A stranded tube may be wound, in one or more concentric clockwise and/or anti-clockwise layer(s), from a plurality of wire threads tightly coiled and pressed together in gapless mutual contact with each other. Stranded tubes HHS may be made of metal such as stainless steel, or Nitinol, or made from non-metallic material, such as a polymer, composite fibers, or other suitable material, or in a combination thereof, and may be coated with a friction-reducing layer of solid or other lubricant, such as Teflon for example, for enhancing smooth operation. Stranded tubes are commercially available. For example, from Fort Wayne Metals, USA, under the name of Helical Hollow Strand, or HHS, which is a Trademark. Details may be found at www.fwmetals.com.

Furthermore, even though being flexible, the prestressed stranded tubes HHS are noted for their outstanding angular torque transmission fidelity.

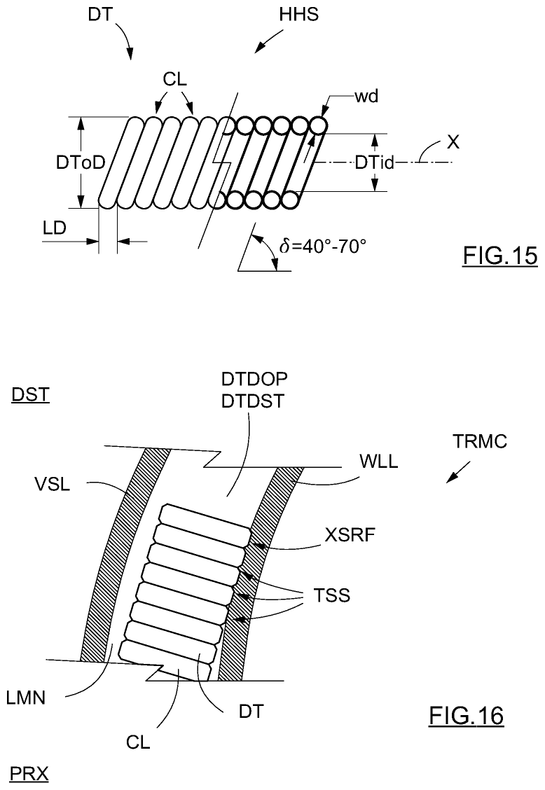

In the embodiments described herewith, the stranded tube of coils HHS shown in FIGS. 14 and 15 are custom made to an exterior diameter DTOD of less than 1 mm, a lumen diameter DTid of less than 0.6 mm, a coil wire diameter wd of about 0.05 mm. The stranded tube of coils HHS may have a winding angle δ of about 40° to 70° relative to the axis X of the stranded drive tube DT. For the sake of angular torque transmission fidelity, the distal end DTDST of the drive tube DT may have more than one, for example two layers of coils wound in the opposite direction relative to each other (one clockwise and the other counterclockwise). More than one layer of coils wound counterclockwise relative to each other also enhance torque transmission in both directions of rotation.

The lumen LMN of a stranded tube HHS, such as the drive tube DT, may be lubricated by solid lubrication, or by hydrophilic lubrication and may be sealed to prevent leaks when conducting fluids or matter such as radiopaque agents or therapeutic agents. With the embodiments described herewith, such agents may be introduced in the drive tube lumen DTLMN with or without retrieving the core wire CRW out of the drive tube proximal opening DTPXO. Those agents may pass from the drive tube open proximal opening to the drive tube open distal opening and thereout via the drive tube lumen DTLMN.

FIG. 16 depicts the distal end of a drive tube DTDST made out of a stranded tube HHS having an exterior surface XSRF supporting a plurality of recessed grooves RCSGR (FIG. 14), which are microgrooves mcGRV provided by the interstices of the prestressed coils CL.

FIG. 16 illustrates an example of how the translation mechanism TRMC of the drive tube DT operates. There is shown a detail of the distal portion of the drive tube deflected arm DTARM, thus of the distal end DTDST of the drive tube DT, which is engaged to progress into a bifurcated vessel VSL1. As described hereinabove with respect to FIGS. 12 and 13, the drive tube distal end DTDST was arrested by friction forces and buckled into the main vessel VSL which prevented proximally delivered push forces to introduce the drive tube deflected arm DTARM into the bifurcated vessel VSL1. For the drive tube deflected arm DTARM to progress into the bifurcated vessel VSL1, the drive tube DT is rotated. Thereby, the coils CL on the exterior surface XSRF of the drive tube DT engage the tissue TSS of the lumen LMN of the bifurcated vessel VSL1, and achieve translation. The same translation mechanism TRMC may apply to the length of the in vivo portion of the drive tube DT.

Evidently, it is the direction of stranding of the coils CL and the direction of rotation of the drive tube DT, either clockwise CW or counterclockwise CCW, that determine the direction of translation of the drive tube DT, either distally DST or proximally PRX.

The rotated distal end DTDST of the drive tube DT will create a pull force to overcome friction forces arresting the distal end DTDST which is then pulled into the bifurcation of vessel VSL1.

There have thus been described mechanisms supported at the distal extremity of a catheter CAT of a catheter apparatus APP, which operates a steering mechanism STMC and a translation mechanism TRMC. The steering mechanism STMC provides a controllable-length deflectable drive tube arm DTARM which is controllable into radial orientation of n×360°, wherein n is an integer. The translation mechanism TRMC for atraumatic rotation-driven translation of the drive tube DT in the lumen of a vessel VSL includes the engagement of the drive tube DT and of the tissue TSS of the vessel VSL, as described hereinabove.

In operation for use, entering into a bifurcated vessel, for example starting from a main vessel VSL to enter into a bifurcated vessel VSL1, may be achieved in three steps.

FIG. 17 refers to a first step, wherein the drive tube DT has been navigated through various vessels, and has reached for example, an initial longitudinal vessel which is selected for the sake of ease of description, as a main vessel VSL. For navigation, the drive tube DT is disposed in the rather flat and straight navigation configuration depicted in FIG. 17, as 211 wherein the drive tube distal open opening DTDOP extends distally away from the nose tip NSTP of the core wire CRW by about 3 cm for example. Thereby, the drive tube distal end DTDST remains soft and flexible since that distal portion of the drive tube lumen DTLMN is empty of core wire CRW and is thus not stiffened thereby. One could also refer to the navigation configuration as a relation to the distance by which the core wire tip CWTP is separated apart from the drive tube distal opening DTDOP. Else, it is possible to say that with the core wire tip CWTP at reference location LOC0 in the drive tube DT, the steering mechanism STMC is disposed in the navigation configuration. In other words, the navigation configuration is independent from the properties of the architecture of the vasculature, or of the furcation, size or angles of branching of the vessels VSL.

It is noted that the rather straight navigation configuration prevents penetration of the drive tube DT into unwanted bifurcations. Furthermore, by the distal portion of the drive tube lumen DTLMN being empty of the core wire CRW, thus not being rigidized thereby, the drive tube distal end DTDST remains soft and flexible, which feature is a safety feature that prevents accidental perforation of a vessel.

In the first step of the navigation configuration, the drive tube DT may thus be navigated along the main vessel VSL to a reference position LOC1 disposed at a predetermined distance away from the furcating vessel VSL1 before being prepared to operate in the second step. The reference location LOC1 shown in FIG. 17 is disposed on the main vessel VSL close to the bifurcated vessel VSL1 desired to be penetrated. The reference location LOC1 is selected in relation with the anatomical properties of the main vessel VS, the bifurcated vessel VSL1 which has to be penetrated, and of the steering mechanism STMC. In other words, the penetration configuration is dependent from the architecture of the vasculature, including size and angle of the related vessels VSL. The first step of operation, in the navigation configuration, is achieved and ends when the drive tube distal end DTDST has reached the reference location LOC1. To proceed to the second step of operation, a reference location LOC2 is required.

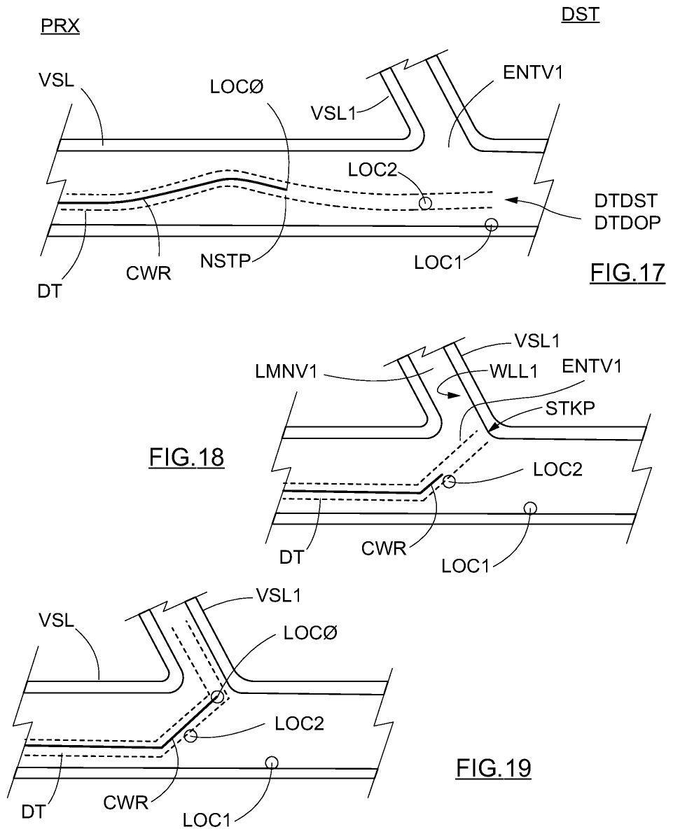

In the second step of operation, the core wire CRW is translated distally along the drive tube DT, out of the navigation configuration reference location LOC0, until the core wire tip CWTP reaches the reference location LOC2 on the drive tube DT. The reference location LOC2 is disposed closer to the drive tube distal opening DTDOP than the reference location LOC0. With the core wire tip CWTP at location LOC2, the core wire CRW is rotated, which also rotates the drive tube DT therewith. The core wire CRW is rotated until oriented in appropriate angular direction aimed at the entry ENTV1 of the bifurcated vessel VSL1, whereby the drive tube deflectable arm DTARM is now able to deflect, as shown in FIG. 18.

FIG. 18 illustrates the disposition of the drive tube DT in the penetration configuration at the end of the second step of operation: The distal arm length DTALN is long enough to reach the bifurcated vessel VSL1 and is well oriented for penetration therein. The drive tube deflected arm DTARM deflects away by an angle α relative to the core wire body portion CWBD. In other words, the drive tube deflected arm DTARM is disposed in contact with a wall WLL1 of the bifurcated vessel VSL1 at least at the entrance ENTV1 thereof, as shown in FIG. 18. The drive tube deflected arm DTARM may be arrested say by friction forces on a wall WLL1 at the entrance ENTV1 of the bifurcated VSL1 and may be stuck at a point STK. Progress of the drive tube DT into the lumen LMNV1 of the bifurcated vessel VSL1 is provided in step 3 by rotation of the drive tube DT, including the drive tube distal portion DTDST.

The various reference locations, namely LOC0, LOC1 and LOC2, may be selected by a practitioner and/or be derived by use of computer programs, such as CAD/CAM programs taking advantage of imaging facilities. The reference location LOC1 and LOC2 shown in FIGS. 17-19 take characteristics of the steering mechanism STMC, such as the core wire bend CWBND and the angle of bend α, in consideration, as well as the properties of the configuration of the vasculature, and of the bifurcations, size or angles of branching of the vessels VSL and VSL1.

At the beginning of step 3, the core wire CRW remains in stationary position relative to the vessel VSL while the drive tube DT progresses into the bifurcated vessel VSL1 as achieved by the rotation thereof in engagement with the tissue TSS of the lumen of the vessel VSL1. With the core wire CRW stationary, progress of the drive tube DT continues until the distance separating apart between the drive tube distal opening DTDOP and the core wire tip CWTP has returned to the navigation configuration, namely to LOC0, as shown in FIG. 19. Thereafter, the core wire CRW and the drive tube DT translate together for the navigation configuration to continue. Once back into navigation configuration as in the first step of operation, the core wire CRW and the drive tube DT are maintained as if in locked in mutual relative disposition. Further loops of steps of operation may now follow sequentially.

There has thus been described a catherization apparatus APP having a catheter portion CAT with a steering mechanism STMC and translation mechanism TRMC. The catheter portion CAT is coupled distally to a tubing portion TUB with tubes and wires, which in turn is coupled to a unit(s) portion UNT, as shown in FIG. 20. The resilient core wire CRW is deformed distally by a core wire bend CWBND to form a core wire nose CWNS which ends in a distal core wire tip CWTP. The drive tube DT has a drive tube lumen DTLMN for holding the core wire CRW therein. The drive tube DT is configured to be disposed and operate in sequence, in one of two configurations. One configuration is the navigation configuration for navigation into body vessels VSL, wherein the core wire bend CWBND is supported in straightened disposition in the drive tube lumen DTLMN. Another configuration is the penetration configuration for entering a bifurcated vessel VSL1, wherein the core wire nose CWNS deflects a distal portion of the drive tube DT into a drive tube deflected arm DTARM.

The drive tube DT of the catheter portion CAT has a drive tube distal opening DTDOP, and in the navigation configuration, the drive tube distal opening DTDOP is disposed distally away from the core wire tip CWTP. Furthermore, the drive tube DT has a drive tube distal opening DTDOP, in penetration configuration, the drive tube DT is configured to operate in two steps. In a first step, the drive tube DT is navigated to a reference location LOC1 adjacent a selected bifurcation vessel VSL1 having a bifurcated vessel opening ENTV1 to be engaged. In a second step, the core wire tip CWTP is disposed at a reference location LOC2 proximally away from the drive tube distal opening DTDOP. Then, the core wire is CRW is turned in radial orientation towards the bifurcated vessel opening ENTV1, whereby the drive tube DT is turned likewise, and whereby the drive tube deflected arm DTARM is deflected for translation into a bifurcated vessel VSL1. The deflected drive tube arm DTARM extends in direction and in continuation of the core wire nose CWNS and distally away from the core wire tip CWTP. The drive tube DT supports microgrooves mcGRV which form a translation mechanism TRMC.

There has also been described a catheterization apparatus APP having a catheter CAT for navigation in a lumen VSLMN of a body vessels VSL. The catheter CAT comprises a flexible drive tube DT having an exterior surface DTSRF supporting helically wound recessed microgrooves mcGRV forming female threads adapted to receive therein tissue TSS from wall of the lumen VSLMN. Thereby, rotation of the drive tube DT into protruding male threads formed by the tissue TSS received in the recessed microgrooves mcGRV drives the drive tube DT into translation. A core wire CRW supported in a lumen DTLM of the drive tube DT and having a distal portion which is deformed a priori into a bend to form a straight distal core wire nose CWNS. The drive tube DT is configured to deflect into a straight arm DTARM following distal translation along the core wire nose CWNS.

Translation of the drive tube DT controls a length DTALN of the deflected arm DTARM. Distal translation of the drive tube DT continues in straight direction away from the core wire nose CWNS. The core wire CRW is configured for controllable radial orientation, whereby the core wire nose CWNS orients the straight drive tube arm DTARM in a same radial orientation.

There has further been described a method for implementing a steering mechanism for a catheter of a catherization apparatus. The method comprises providing a core wire distally deformed by a bend, and providing a drive tube having a drive tube lumen holding the deformed core wire therein, whereby translation of one out of the core wire and the drive tube relatively to each other allows disposing the steering mechanism in one out of a navigation configuration and a penetration configuration.

The method also comprises providing translation mechanism operating microgrooves disposed on the exterior surface of the drive tube to engage lumen tissue when the drive tube is rotated. Rotation of the drive tube rotates the drive tube distal end for providing traction force for translation into a bifurcated vessel. The drive tube has a drive tube lumen via which radiopaque agents and therapeutic agents may be communicated from a drive tube proximal opening to a drive tube distal opening and thereout.

It will be clear to a person skilled in the art that the control of the drive tube and of the core wire can be either manually operated by the user or through a motorized and computerized control unit. Such a control unit can be controlled by the user and allow for a more precise control of the displacement and rotational motions. In addition, in some preferred embodiments an algorithm receiving as input the image of the path through which the drive tube needs to advance as well as the location of the bifurcation points along that path will be able to calculate in advance the combined optimal parameters for approaching each bifurcation. In further embodiments, the target point can be marked on the image and the algorithm will detect each bifurcation and calculate optimal path as well as required parameters for each bifurcation. In an additional embodiment one of the algorithms described above can be combined in the control unit so as to automate the planning and performance of the procedure in whole or in part, while optionally providing a simulation facility for the user.

### Relative Bending Stiffness

The deflection of the drive tube distal end DTDST may be achieved differently from the description hereinabove, but it is still the relative disposition of the core wire CW and of the drive tube DT that is operative to control that deflection.

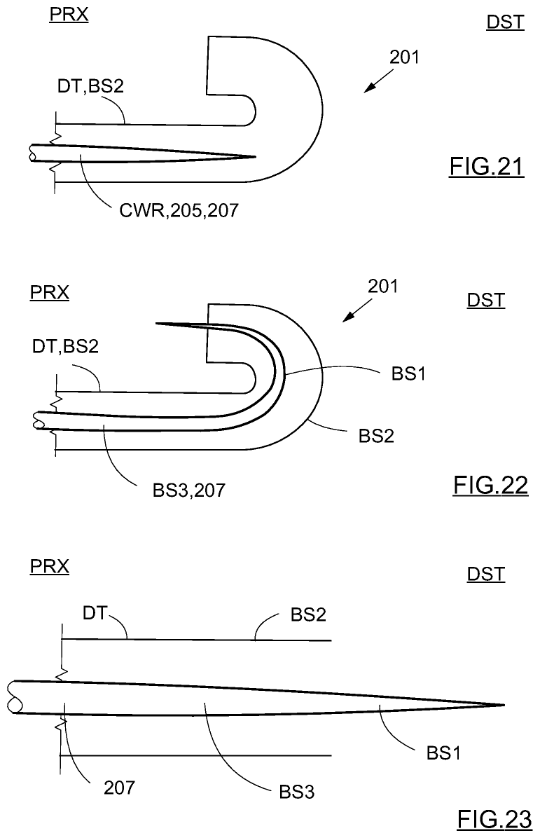

FIG. 21 depicts a drive tube DT which supports a core wire CW therein. The core wire CW may have a bending stiffness BS that varies along the length thereof, either monotonously, or abruptly, or according to a predetermined distribution of values. In FIG. 21, the drive tube DT has a deformed distal end DTDST, which is referred to as a distal initial bend 201, or initial bend 201. Such a distal initial bend 201 may include for example, a curvature selected as a hairpin-curve, a semicircular curve, a J-shaped curve, a U-shaped curve, or an elliptic curve.

In FIG. 21, the core wire CWR is shown as a slender cone, to illustrate that the bending stiffness BS thereof is not longitudinally constant, but increases from nil at the core wire tip CWTP or 205, and grows to a larger bending stiffness value along the proximal direction PRX. Such a core wire CWR is thus a variable stiffness core wire 207. In practice, a distribution of bending stiffness BS may be achieved by coating a slender conically shaped core wire CWR with a medical regulations compatible plastic material, such that a core wire CWR of longitudinally constant diameter is achieved.

Contrary thereto, the drive tube DT may have a bending stiffness BS2 of constant value. That constant value bending stiffness BS2 may be superior to the bending stiffness BS1 of the distal DST portion of the variable stiffness core wire 207. Furthermore, that constant value bending stiffness BS2 may be inferior to the bending stiffness BS3 of the proximal portion PRX of the variable stiffness core wire 207.

FIG. 22 depicts the drive tube DT wherein a distal portion of the variable stiffness core wire 203 has been translated in engagement in the distal initial bend 201. The shape of the curvature of the initial bend 201 will evidently not change as long as the constant bending stiffness BS2 of the drive tube DT is higher than the lesser bending stiffness BS1 of the distal portion of the variable stiffness core wire 207.

FIG. 23 shows the drive tube DT wherein a portion of the variable stiffness core wire 207 having a bending stiffness BS3 that is higher than the constant bending stiffness BS2 of the drive tube DT has been translated in engagement with the initial bend 201. This time, the higher bending stiffness BS3 of the core wire CWR has redressed and unbent the initial bend 201. The drive tube DT is now longitudinally straightened out in navigation mode 211 for translation into the vasculature, and proximal retraction of the variable stiffness core wire 207, relative to the drive tube DT, will redress the curvature of the initial bend 201 of the drive tube distal end 203. It is noted that the transition between the initial bend 201 and the longitudinally straightened out in navigation mode is controllable, still as result from the relative translation between the variable stiffness core wire 207 and the drive tube DT.

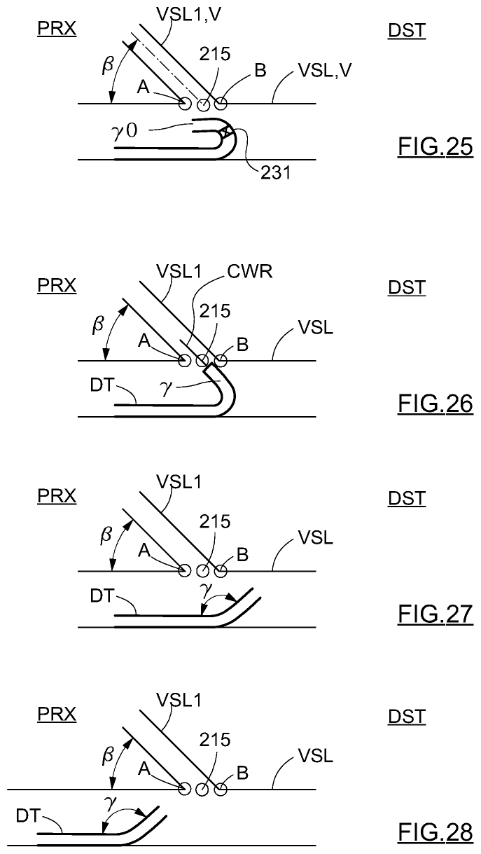

FIG. 24 illustrates a drive tube DT with an initial bend 201 when disposed in a main vessel VSL wherefrom a proximally oriented bifurcation vessel VSL1 extends. For the sake of clarity, the variable stiffness core wire 207 supported in the lumen LMN of the drive tube DT is represented by the axis XL. The controllable clockwise deployment of the curvature of the distal bend 201 into the straightened-out navigation mode 211 orients the opening of the initial bend 209 which deflects away from facing the proximal direction PRX to face the distal direction DST, shown by the deflection angles, marked respectively as γ0 and γ4. The angles γ are measured between the axis X of the vessel VSL, which axis is not shown, to the axis XL. Therefore, γ0=0°, and γ4=180°. The opening of the initial bend 209 thus points in a direction of orientation which forms an angle γ with the X-axis. For example, the deployed curvatures marked γ1, γ2, and γ3, form an angle of about, respectively, 45, 90, and 135 degrees. The angle γ may thus practically cover a range of zero to 180 degrees, from an acute angle γ to an obtuse angle γ. The angle γ which is measured in the same direction as the therefrom different angle β shown in FIGS. 25 to 28, which angle β indicates the angle of orientation of a bifurcating or branching vessel VSL1.

It is thus the relative bending stiffness BS portions or segments of the drive tube DT and the variable stiffness core wire 207 that permit to control the direction of orientation of the opening of the initial bend 209, or axis XL of the lumen.

FIGS. 25 to 28 present a schematic simplified cross-sectional illustration for the description of how the distal tube DT may be engaged for introduction in a bifurcating vessel VSL1, which slants in proximal direction PRX, and makes an acute angle β with a main vessel VSL extending distally away.

In FIG. 25, the drive tube DT has been translated in a lumen LMN of a vessel VSL, from a proximal to a distal direction, relative to a branch opening 215 of an out of the main vessel VSL bifurcating vessel VSL1. After having reached a preplanned disposition relative to the branch opening 315, the catheter is operated to regain the shape of the initial bend 201. The initial bend 201 is imaged in plan projection disposition to clearly discern the true measure of the angle γ and the true direction of orientation of the drive tube opening of the initial bend 209. In practice during operation, the true measure of the initial bend 209 is easily distinguished on an image: It suffices to appropriately rotate the drive tube DT to obtain the desired true measure of the initial bend 209. However, radiopaque markers 231, disposed on the drive tube DT may be used to facilitate the task of the practitioner P, and may be beneficial to discern lengths along the catheter CAT, and to distinguish between portions and even angular rotation measures of the drive tube DT.

FIG. 25 also shows the angle β of the bifurcating vessel VSL1 relative to the main vessel VSL as well as the rim corners A and B, seen in two-dimensional projection as the intersection of the branch opening 215 with the plane of projection. The curvature of the drive tube distal end 203 still has the shape of the distal initial bend 201.

In FIG. 26 the distal initial bend 201 is shown to have been partially deployed counterclockwise. A portion of the variable stiffness core wire 207 protrudes out of the drive tube open end 203. The drive tube DT is shown to be properly disposed to engage the branch opening 315 after short translation steps intended to make contact with tissue of the bifurcating vessel VSL1. Once the microgrooves mcGRV of the drive tube DT are engaged in tissue TSS of the bifurcating vessel VSL1, is suffices to rotate the drive tube DT for this last one for penetrate in screw-like progression in the lumen LMN of the branching vessel VSL1.

FIG. 27, like FIG. 26, illustrates the deployment of the distal initial bend 201, which this time has been deployed too far distally away from the branch opening 215. Proximal translation may cause a spring back of the drive tube DT and return to the disposition shown in FIG. 26. In case of failure, the drive tube DT may be driven into navigation mode 211 and another penetration effort may be attempted.

FIG. 28, like FIG. 26, illustrates the deployment of the distal initial bend 201, which this time has been performed too far proximally away from the branch opening 215. Distal translation may cause a spring back of the drive tube DT and return to the disposition shown in FIG. 26. In case of failure, the drive tube DT may be driven into navigation mode 211 and another penetration effort may be attempted.

Similarly to the variable stiffness core wire 207, the drive tube DT too may be configured as variable stiffness drive tube 221. Thereby, a higher bending stiffness of the drive tube DT will prevail over a thereto relative lower bending stiffness of the variable stiffness core wire 207. This means that it is possible to use the variable stiffness drive tube 221 to deform the variable stiffness core wire 207, instead of the contrary which is described hereinabove in relation to FIG. 3 for example.

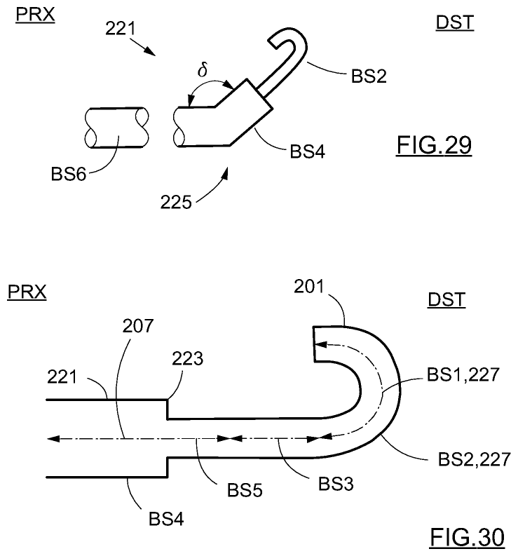

FIG. 29 shows a variable stiffness drive tube 221 with a flexible redressible drive tube bend 225 which is elastically bent at an angle δ, similarly to the bending of the core wire CWR in FIG. 3. The distal portion of the variable stiffness drive tube 221 may have a bending stiffness of value BS2 and a proximal portion thereof may have a bending stiffness of value BS4 which is more rigid than the value BS2. To emphasize that BS4>BS2, the portion of the variable stiffness drive tube 221 having a bending stiffness of value BS4 is shown in exaggerated size relative to the distal portion marked BS2. If desired, the variable stiffness drive tube 221 may further have a portion thereof which is proximal to the portion marked BS4 and has a bending stiffness marked BS6 that is superior to the bending stiffness of value BS4. In practice, with stranded tube of coils HHS a shown in FIGS. 14 and 15, the bending stiffness is controllable, for example by winding the tube with more than one layer of coils. For control of the bending stiffness BS, a second layer of coils may be added, made of the same material or a material which is different from that of the first layer of coils, or the second layer may simply have spaced apart coils.

FIG. 30 is a schematic representation used to illustrate the deformation capability of a variable stiffness drive tube 221 operating in association with a variable stiffness core wire 207. A distal portion of a variable stiffness drive tube 221 having a distribution of different bending stiffness values BS is depicted, indicating for example, that a drive tube initial bend 201 with a bending stiffness value of BS2 which extends proximally away up to a portion thereof having a bending stiffness BS4, indicated to start “virtually” at a step 223 having a value of BS4 which extends proximally away. A variable stiffness core wire 207 with a distribution of different bending stiffness values BS is shown to start from the core wire tip 205 at the value of BS1, and extends proximally PRX through the bending stiffness values of BS3 and BSS. In FIG. 30, the bending stiffness values increase arithmetically from the lowest value BS1 to the highest value BSS. A portion of length or zone of same bending stiffness BS is indicated as 227 in FIG. 30.

As described hereinabove, the distal translation of the variable stiffness core wire 207 having a bending stiffness value of BS3 will deflect the distal initial bend 201 since BS3 >BS2. With a variable stiffness drive tube 221 having a flexible drive tube bend 225, as shown in FIG. 29, of variable stiffness length, shown in FIG. 30 to have values of BS2 and BS4 remain aligned by being supported by a portion of the variable stiffness core wire 207 having a larger bending stiffness value of BS5.

However, when the variable stiffness core wire 207 is retracted proximally PRX away for the portion thereof marked BS5 to become proximal to the drive tube bend 225, shown in FIG. 29, of the variable stiffness drive tube 221, the drive tube bend 225 will become free to extend in erection.

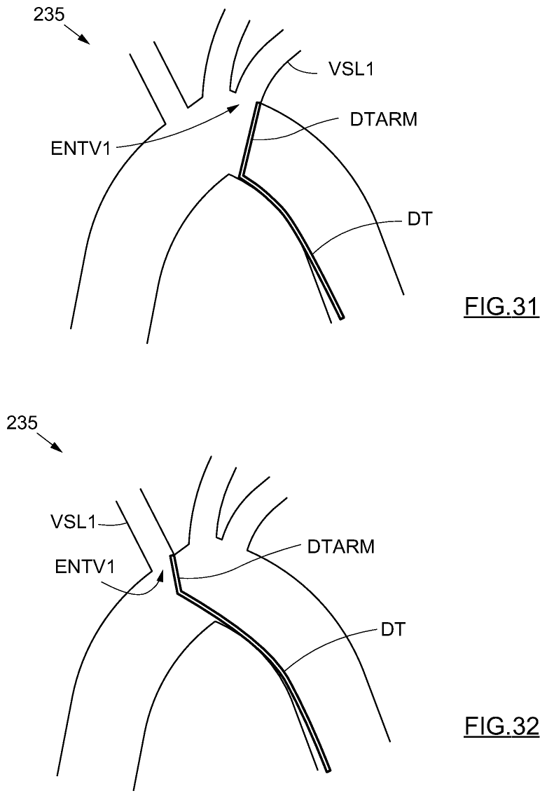

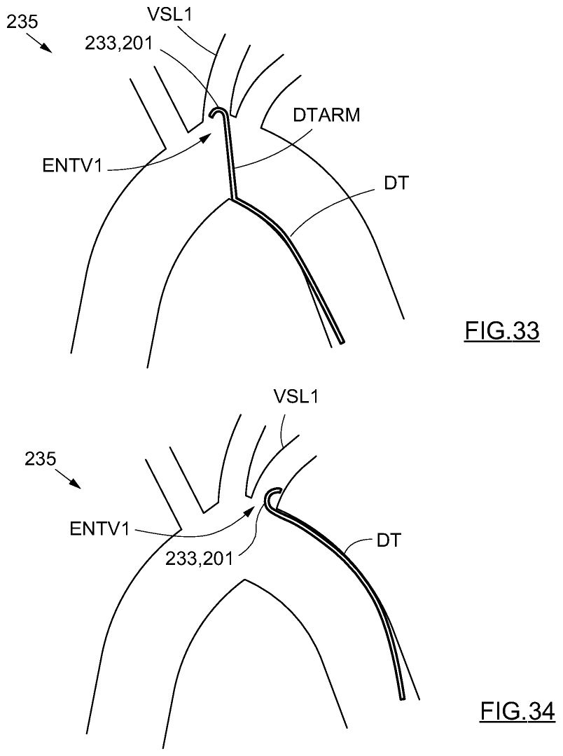

FIGS. 31 to 35 refer to the catherization of aortic type III arch bifurcations.

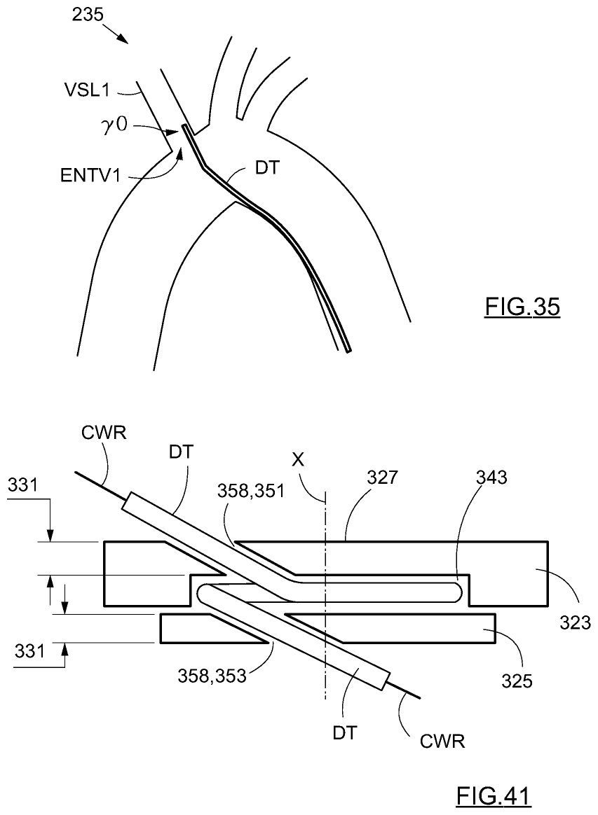

In FIGS. 31 and 32, the drive tube DT which supports a core wire CRW, not shown, having a core wire bend CWBNB therein, as in FIG. 6, is shown after having been navigated in disposition relative to a bifurcation VSL1 and having made contact with the entry ENTV1 thereof. For this procedure, the drive tube DT has been navigated to a first reference location LOC1, shown in FIG. 17, with the drive tube distal opening DTDOP extending distally away from a nose tip NSTP of the core wire CRW, as shown in FIG. 17. Then, the nose tip NSTP, still in FIG. 17, was translated to a second reference location LOC2, shown in FIG. 17, where core wire CRW was translated in appropriate position for erection of the drive tube arm DTARM to deflect away.

Subsequently, the drive tube DT was translated over the core wire CRW and away therefrom, to grow first a short length DTLN, shown in FIG. 8, of drive tube arm DTARM. Next, both the drive tube DT and the core wire CRW were rotated and oriented in appropriate angular direction aimed at the entry ENTV1 of the bifurcation VSL1. In turn, the drive tube DT was translated along the core wire CRW to grow a desired length DTLN, for engagement thereof with the entry ENTV1 of the bifurcation VSL1.

Finally, with the drive tube DT in contact with the entry ENTV1 of the bifurcation VSL1, it remains to translate the core wire CWR out of the drive tube DT and into the bifurcation VSL1 whereafter the drive tube DT is translated over the core wire CWR for further navigation in the bifurcation VSL1.

In FIGS. 33 and 34, the drive tube DT supports therein a first core wire CRW, not shown, having a core wire bend CWBNB as in FIG. 6, and a distal initial bend 201.

The drive tube DT is shown after having been navigated in disposition relative to a bifurcation VSL1 and having made contact with the entry ENTV1 thereof. For this procedure, the drive tube DT was navigated to a first reference location LOC1, shown in FIG. 17, with the drive tube distal opening DTDOP extending distally away from a nose tip NSTP of the core wire CRW, as shown in FIG. 17, where core wire CRW was translated in position for erection of the drive tube arm DTARM to deflect away. Thereafter, the drive tube DT is translated over the core wire CRW and away therefrom, to grow a short length DTLN of drive tube arm DTARM, and next, both the drive tube DT and the core wire CRW are rotated together until the drive tube arm DTARM is oriented in appropriate angular direction aimed at the entry ENTV1 of the bifurcation VSL1. Next, the drive tube DT is translated along the core wire CRW to grow a desired length DTLN, for engagement and contact thereof in the entry ENTV1 of the bifurcation VSL1.

Once the drive tube DT is disposed in engagement and support thereof by the bifurcated vessel VSL1, the first core wire CWR is retrieved proximally out of the drive tube DT and is replaced by a second core wire 207 supporting a plurality of portions of length 233 having different values of bending stiffness BS, wherein at least one of which has a value superior to the bending stiffness BS value of the initial bend 201,

The second core wire 207 is drive in translation into the drive tube DT and through the distal initial bend 201 for one out of the plurality of portions of length 233 having a bending stiffness BS value superior to the bending stiffness BS value of the initial bend 201, to deform the initial bend 201 in straightened out disposition as shown in FIG. 35. This means that the distal initial bend 201 has an angle γ equal to zero, as shown in FIG. 24.

At this stage, the drive tube may progress into the bifurcated vessel VLS1 by use of one or more of translation over a thereout extended core wire CWR and of rotation of the drive tube DT.

### Actuation Device

Ex vivo problems encountered with catherization include cumbersome handling of long and thin resilient microcatheter tubing and wires, as well as the critical need for precise control of desired motions of translation and rotation of those tubing and wires. To mitigate the cumbersome handling, it seems best to orderly coil the microcatheter tubing for ease of operation.

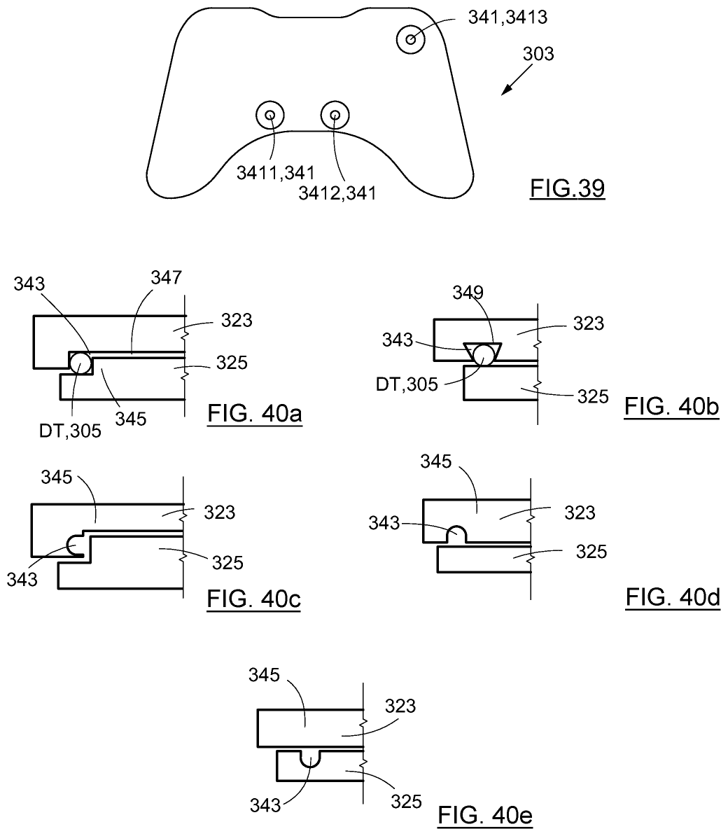

With respect to the critical need of precision, which is not shown in the figures, there is provided a command post 301, shown in FIG. 36, well equipped with three-dimensional imaging facilities and three-dimensional computation program facilities for planning and performing catherization. From the command post 301, the practitioner operates a control station 303 to deliver precise commands of desired motions to be performed by the microcatheter 305, in response to from in vivo received images and feedback data. Such desired motions include translation and rotation of the drive tube DT and translation and rotation of the core wire CWR provided by computer programs as sub-millimeter lengths and sub-degree rotation values. Those precise commands are conveyed to a remotely controlled actuation device 307. It is from the actuation device 307 that the microcatheter 309 is actuated for translation and rotation of the drive tube DT and of the core wire CW. The actuation device 307 includes a rotatable turntable 311, shown in FIGS. 37 and 38, which supports at least a plurality of actuators 313. The remote-control transmitter or transceiver 317, the microelectronics, wiring and power supply for operation of the actuation device 307 may be disposed on each one of the table top disk 323, or on the base disk 325, or be distributed between both of the two concentric disks 321.

FIG. 36 is a schematic illustration for ease of orientation in the catherization surrounding environment. Distally DST, a guiding catheter GC has been inserted by the practitioner in the patient P, and for planning the intervention, available 3D-imaging and 3D computer programs are provided in a well-equipped command post 301. The command post 301 is a portion of the unit UNT, also shown in FIG. 20, with catherization intervention support, and includes equipment, manpower, hardware, and computer programs, shown to be disposed proximally PRX. Units UNT are well known to those skilled in the art. Next, the actuation device 307 is provided already loaded with a microcatheter 305, with or without a symbolically shown Y-connector coupling, the actuators 313, a remote-control transmitter or transceiver 317, and a power supply such as a rechargeable battery. The Y-connector Y is shown only symbolically in FIG. 36. Then, the distal portion of the microcatheter 305 is engaged with the Y-connecter coupling Y, which is coupled to the guide catheter GC already inserted in the patient P.

The actuation device 307 is a construction similar to a turntable 319, which includes two concentric disks 321 which are mutually coupled for rotation about an axis X, by means of a machine bearing 315 for example. The two disks 321 include a table top disk 323 which is disposed on top of and rotates concentrically relative to a base disk 325. Each one of the disk 321 has a disk top surface 327, a disk bottom surface 329, and a disk thickness 331. The table top disk 323 supports a plurality of actuators 313 configured to apply rotation and translation to each one of the drive tube DT and to the core wire CWR.

FIG. 38 depicts a top view of the table top disk 323. It is on the disk top surface 327 of the table top disk 323 that the actuators 313 are disposed. In one embodiment, the actuators 313 may include a core wire rotation actuator 333, a core wire translation actuator 335, a drive tube rotation actuator 337, and a base disk actuator 339. This last base disk actuator 339 spins the top disk 323 of the turntable 311 via a base disk motorized driver 355, such as a rotating roller for example. Furthermore, the actuation device 307 also includes a remote-control transmitter or transceiver 317, and a power supply.

FIG. 39 illustrates an exemplary embodiment of a handheld manually operated control station 303, shown in top elevation, for remote control of the actuators 313. The control station 303 may support three joysticks 341, namely a first joystick 3411, a second joystick 3412, and a third joystick 3413. The actuators 313 may be operated by the joysticks 341 in ON and OFF dispositions, at controllably selected speeds and at predetermined preset speeds. Translation of the drive tube DT is performed by rotation of the turntable 319 by the base rotation actuator 319. The drive tube DT is clamped to the top disk 323 which rotates and thereby discharges the portion of drive tube DT out of the channel 343 and distally away.

The following commands, transmitted, by the control station 303 to the actuators 313, are the result of the displacement of the first joystick 3411 in the following directions:
Forward: Core wire CW advancement at controlled speed.
Backward: Core wire CCW retraction at controlled speed.
Right: Core wire CW rotation at constant slow speed.
Left: Core wire CCW rotation at constant slow speed.

The following commands, transmitted by the control station 303 to the actuators 313, are the result of the displacement of the second joystick 3412 in the following directions:
Forward: Drive tube advancement at constant slow speed.
Backward: Drive tube DT retraction at constant slow speed.
Right: Drive tube DT CW rotation at constant slow speed.
Left: Drive DT CCW rotation at constant slow speed.

The following commands, transmitted by the control station 303 to the actuators 313, are the result of the displacement of the third joystick 3413 in the following directions:
Forward: Microcatheter 305 advancement at controlled speed.
Backward: Microcatheter 305 retraction at controlled speed.

FIG. 37 schematically illustrates additional features of an exemplary embodiment of the actuation device 307 and of the turntable 311. The turntable 311 includes two concentric disks 321 which are mutually coupled for rotation about an axis X. The two disks 321 include a table top disk 323 which is disposed on top of and rotates concentrically relative to a base disk 325. To store a portion of length of the microcatheter 305 therein, a channel 343 is created between the concentric disks 321.

The channel 343 may be formed between a circular protrusion 345 extending out of a top surface 327 of the base disk 325 which is concentric to and penetrates into a circular recess 347 entered into the bottom surface 329 of the top disk 323. Laterally, the channel 343 is formed by the difference between the smaller exterior diameter of the protrusion 345 and the larger interior diameter of the recess 347. Height wise, the channel 343 is formed by the distance of separation between the bottom of the recess 347 and the top of the protrusion 345. In the example shown in FIG. 37, the cross-section of the channel 343 is square or rectangular, with two sides thereof pertaining to the table top disk 327 and the other two sides being part of the base disk 327. The sides of the cross-section of the channel 343 may not be straight and at least one side thereof pertains to the table top disk 323 and at least one other side thereof pertains to the base disk 325. A preferred cross-section has a trapezoidal shape 349 and the shorter of the two parallel sides thereof is provided by the base disk 325.

FIGS. 40a through 40e illustrates a few exemplary embodiments of the cross-section of the channel 343. In FIG. 40a, two sides of the cross-section of the table top disk and of the base disk 325 are in contact with the drive tube DT. FIG. 40b shows the preferred embodiment, and FIGS. 40c, 40d and 40e present cross-sections of the channel 343 having a rounded-off channel side.

FIG. 41 illustrates the looped path of the microcatheter 305 into and out of the turntable 311. The channel 343 is configured to rigidly and orderly support, guide, and orient the flexible microcatheter 305 for unhindered passage in vivo, even when pushed out of the turntable 311 in distal direction DST, or when retracted therein by being pulled in proximal direction PRX. The drive tube DT supporting the core wire CWR therein, passes via a top groove 351 opened in the disk top surface 327 of the table top disk 323 and via a passageway 358, into the channel 343. The top groove 351 is cut on top of and in conformance with the channel 343, and leads the microcatheter 305 in gentle monotonous slope into the channel 343. It is from the top groove 351 and via the passageway 358 that the drive tube DT penetrates into the circular channel 343 which is created by and between the two disks 321. Similarly, the drive tube DT exits out of the channel 343 through a passageway 358 and a base groove 353 which is opened in the disk bottom surface 329 of the base disk 325.

The channel 343 is disposed concentrically and close to the periphery of the table top disk 323 to reach a length as long as practically possible, so as to be able to receive therein of a relatively long portion of the drive tube DT. For example, with a channel 343 having a diameter of 19 cm, the length of the microcatheter 305 stored in the channel 343 is about 60 cm, whereby the turntable 311 may have a diameter of about 20 cm. The microcatheter 305 thus may exit out of turntable 311 after having covered at most, almost a complete circular loop in the guiding channel 343. Hence, the actuation device 307 is configured to support and guide the microcatheter 305 along a controlled drive tube length which may be short but may span up to a maximum of about 60 cm. That controllable drive tube DT length extends between the exit out of passageway 358 of the top disk 323 up to the passageway 358 in to the base disk. The length of the portion of the drive tube DT supported by the channel 343 is controllable. The channel 343 provides rigid mechanical backing support to push the microcatheter 305 in vivo. The drive tube DT is confined in the channel 343 in rigid support to prevent buckling and/or deformation thereof.

There has thus been described a catheter CAT wherein each one of the drive tube DT and the core wire CRW supports a distribution of portions of length 233 having a bending stiffness BS of different value, whereby relative mutual disposition of the portions of length 233 having a bending stiffness BS of different value pertaining to the drive tube DT and to the core wire CRW produces a reversible controlled deformation of at least one of the drive tube DT and the core wire CRW. It is the relative translation of the drive tube DT and of the core wire CRW that commands a controllable extension of the deformation of shape. The drive tube DT has a distal initial bend 201, and the relative mutual translation between the drive tube DT and the core wire CRW commands controlled reversible deployment of the initial bend 201. Further, the drive tube DT has a distal initial bend 201 ending in a drive tube distal end DTDST, and relative mutual translation between the drive tube DT and the core wire CRW commands controlled reversible direction of orientation of the drive tube distal end DTDST.

The drive tube DT supports at least one flexible redressible bend 225, and the relative mutual translation between the drive tube DT and the core wire CRW commands controlled disposition of the bend 225 in one of a straightened-out disposition and a deflected disposition. The controlled disposition by relative mutual translation of the drive tube DT and the core wire CRW commands a reversible deformation of shape of the drive tube DT and of the core wire CRW. A radiopaque marker may be applied on at least one portion of length 233 of at least one of the drive tube DT and the core wire CRW to indicate a value of bending stiffness BS, as well as a radial orientation and a measure of length. Radiopaque markers may be applied to the drive tube DT and of the core wire. A core wire CRW having a plurality of portions of length 233 having a bending stiffness BS of different value is configured to reversibly deploy a distal initial bend 201 having a bending stiffness BS of lower bending stiffness value than one of the plurality of portions of length 233.

A method for implementing a catheter CAT for providing each one of the drive tube DT and the core wire CRW with a plurality of portions of length 233 having a bending stiffness BS of different value, and operating the plurality of portions of length 233 in relative mutual translation to command a controlled reversible deformation of shape of at least one of the drive tube DT and the core wire CRW. The method wherein a portion of length 233 is one of a segment or a portion of length 233 having a definite bending stiffness BS, and a segment of specific length 233 having a monotonously changing bending stiffness BS with a peak bending stiffness BS. The method wherein the core wire CRW has a plurality of portions of length 233 having a bending stiffness BS of different value, and the drive tube DT has a distal initial bend 201 which is reversely deployable in controlled angular disposition by relative mutual translation of the drive tube DT and the core wire CRW. The method wherein the drive tube DT is reversibly and controllably redressed from the straightened-out disposition into a selected angular disposition.

A method for penetration into an aortic type III arch bifurcation VSL1 wherein the drive tube DT, which supports therein a core wire CWR having a core wire bend CWBNB, is navigated to a first reference location LOC1, with the drive tube distal opening DTDOP extending distally away from a nose tip NSTP of the core wire CRW, and wherein the nose tip NSTP is translated to a second reference location LOC2 from where core wire CRW is translated for erection of the drive tube arm DTARM which as result thereof, deflects away, and following which, the drive tube DT is translated over the core wire CRW and away therefrom, to grow a desired length DTLN of drive tube arm DTARM, and next, both the drive tube DT and the core wire CRW are rotated together until the drive tube arm DTARM is oriented in appropriate angular direction aimed at the entry ENTV1 of the bifurcation VSL1.

A method for penetration into an aortic type III arch bifurcation wherein the drive tube DT supports a distal initial bend 201 and a plurality of portions of length 233 having different values of bending stiffness BS, wherein at least one portion 233 of which has a bending stiffness BS which has a value superior to the bending stiffness BS value of the initial bend 201. The method includes a first core wire CWR, having a core wire bend CWBNB, which is supported in the drive tube DT and which is navigated to a first reference location LOC1, with the drive tube distal opening DTDOP extending distally away from a nose tip NSTP of the core wire CRW, wherein the nose tip NSTP is translated to a second reference location LOC2 and wherein core wire CRW is translated in position for erection of the drive tube arm DTARM to deflect away, following which the drive tube DT is translated over the core wire CRW and away therefrom, to grow a desired length DTLN of drive tube arm DTARM, and next, both the drive tube DT and the core wire CRW are rotated together until the drive tube arm DTARM is oriented in appropriate angular direction aimed at the entry ENTV1 of the bifurcation VSL1. The method further includes the translation of the drive tube DT along the core wire CRW to grow a desired length DTLN, and is disposed for engagement and support at or into the entry ENTV1 of the bifurcation VSL1, wherein the first core wire CWR is retrieved out of the drive tube DT and is replaced by a second core wire 207 supporting a plurality of portions of length 233 having different values of bending stiffness BS, wherein at least one of which has a value superior to the bending stiffness BS value of the initial bend 201. Next, the second core wire 207 is driven in translation into the drive tube DT and through the distal initial bend 201, for one out of the plurality of portions of length 233 having a bending stiffness BS value superior to the bending stiffness BS value of the initial bend 201, to deform the initial bend 201 in straightened out disposition.

An apparatus APP comprising a microcatheter 305 including a drive tube DT which supports a core wire CRW therein, and an actuation device 307 having a rotatable disk 323 which is configured to provide mechanical support and motion to the microcatheter 305, whereby actuation orders, delivered by a control station 303 which is coupled in communication with the actuation device 307, controls translation and rotation of the drive tube DT and of the core wire CRW. The apparatus APP wherein the actuation device 307 orderly dispenses, retracts, and guides a predetermined and controlled length of up to at least 60 cm of the microcatheter 305 in response to actuation commands received from the command post 301. The apparatus APP wherein the command post 301 operates the actuation device 307 by remote control.

The apparatus APP wherein the actuation device 307 supports a plurality of actuators 313 configured to bidirectionally translate and bidirectionally rotate each one of the drive tube DT and the core wire CRW, at a rate of precision of, respectively, sub-millimetric translation and sub-degree rotation. The apparatus APP wherein the actuation device 307 provides a rigid guiding channel to mechanically support the microcatheter in buckling-free and in entanglement-free orderly disposition. The apparatus APP wherein the actuation device 307 is further configured as a rotatable turntable 311 having a diameter of about 15 cm to 25 cm, preferably of about 15 cm to 22 cm, and more preferably of about 16 to 19 cm. The apparatus APP wherein the guiding channel 343 is concentric and close to a periphery of the rotatable turntable 311. The apparatus APP wherein the drive tube DT is enclosed and is stiffly and rigidly mechanically supported in the guiding channel 343, and each one of the drive tube DT and the core wire CRW is translatable and rotatable in the guiding channel 343.

The apparatus APP wherein the microcatheter 305 is driven into translation by rotation of the turntable 311. The apparatus APP of claim 37, wherein the drive tube DT of the microcatheter 305 is driven into translation by rotation of the turntable 311.

The apparatus APP wherein rotation of the turntable 311 drives a controlled length of the drive tube DT in distal direction DST by forces applied for distal penetration into a target vessel VSL, and the guiding channel 343 is configured to mechanically support and guide therein of the controlled length in buckling-free and in entanglement-free guiding channel compliant disposition. The apparatus APP wherein the actuation device 307 is packaged as a disposable throwaway assembly.

A method is provided for implementing a catheterization apparatus APP, comprising a catheter CAT including a drive tube DT and a core wire CWR, for navigation through sinuous body vessels VSL, the apparatus APP comprising three-dimensional imaging facilities and three-dimensional support facilities including computerized command and control of the microcatheter CAT. The apparatus APP provides a turntable 311 supporting a channel 343 for mechanically confining and supporting a distal portion of the catheter CAT therein, wherein a rotational motion is provided to the drive tube DT to enhance distal translation thereof into a target vessel VSL, and arresting motions of the core wire CWR relative to the target vessel VSL while the turntable 311 drives the drive tube DT into the target vessel VSL, such as a bifurcating vessel VSL.

A method PP including a catheter CAT for navigation through sinuous body vessels VSL wherein the catheter includes a drive tube DT having a lumen LMN supporting a core wire CWR therein; the catheter being operative for penetrating into a bifurcating target vessel VSL1 forming an angle with a main vessel VSL. The method comprises providing computer data from a unit portion UNT to a control station 303 for transmission to an actuation device 307. The method further comprises providing the actuation device 307 with actuators 313 and with a channel 343 for support of the catheter along a controlled portion of length of the channel 343, and for operation of the actuators 313 according to data from the unit portion UNT. In addition, the method also comprises operating the actuation device 307 for driving the catheter CAT into a target vessel VSL and for operating according to data received from the unit portion UNT.

A method for implementing a catheter CAT with a drive tube DT and a core wire CWR, with facilities supporting three-dimensional imaging facilities and three-dimensional computer programs, wherein the catheter CAT is operated by digital computerized command and control.

The embodiments described hereinabove are applicable in the medical devices producing industry.

1. A catheterization apparatus including a catheter for navigation through body vessels, the catheter comprising:
a steering mechanism including a drive tube and a resilient straight core wire deformed distally at an angle into a core wire bend to form a transition between a core wire body portion and a straight core wire nose which ends in a distal core wire tip, and
a drive tube having a drive tube lumen holding the core wire therein,
wherein the steering mechanism is configured to be changeable between configurations including: (i) a navigation configuration for navigation in bodily vessels, in which the drive tube lumen is in a straightened disposition supporting the core wire bend therein, and (ii) a penetration configuration for entering a bifurcated vessel,
wherein the core wire nose is configured to deflect a distal portion of the drive tube into a straight drive tube deflected arm oriented in continuation past the core wire tip,
wherein the drive tube has a drive tube distal opening, and
wherein the catheterization apparatus is configured to engage a bifurcated vessel opening by: first, navigating the drive tube distal opening a reference location relative to the bifurcated vessel opening to be penetrated, second, driving the core wire tip to a second reference location which is disposed proximally away from the drive tube distal opening, and third, rotating the core wire into a radial orientation towards the bifurcated vessel opening, which also rotates the drive tube which is configured to be translated over the core wire to create the straight drive tube arm.

2. The apparatus of claim 1, wherein the drive tube comprises microgrooves on an exterior surface thereof that are engageable with lumen tissue.

3. The apparatus of claim 2, wherein rotation of the drive tube also rotates the drive tube distal end to provide traction force for translation into the bifurcated vessel.

4. The apparatus of claim 1, wherein the apparatus comprises a catheter portion including the catheter, a tubing portion, and a unit portion.

5. The apparatus of claim 1, wherein a radiopaque marker is applied on at least one portion of length of at least one of the drive tube and the core wire to indicate a value of bending stiffness.

6. The apparatus of claim 1, wherein each one of the drive tube and the core wire includes a distribution of portions of length having a bending stiffness of different value, whereby relative mutual disposition of the portions of length of the drive tube and the core wire having bending stiffnesses of different value produces a reversible controlled deformation of at least one of the drive tube and the core wire.

7. The apparatus of claim 1, wherein at least one of the drive tube and the core wire includes a plurality of portions of length having different bending stiffness values, whereby relative mutual translation of the drive tube and the core wire commands a reversible deformation of shape of one of the drive tube and the core wire.

8. The apparatus of claim 7, wherein relative translation of the drive tube and of the core wire causes a controllable extension of the deformation of shape of at least one of the drive tube and the core wire.

9. The apparatus of claim 7, wherein:
the drive tube has a distal initial bend, and
relative mutual translation between the drive tube and the core wire causes controlled reversible deployment of the initial bend.

10. The apparatus of claim 7, wherein:
the drive tube has a distal initial bend ending in a drive tube distal end, and
relative mutual translation between the drive tube and the core wire commands controlled reversible direction of orientation of the drive tube distal end.

11. The apparatus of claim 7, wherein:
the drive tube supports at least one flexible bend, and
relative mutual translation between the drive tube and the core wire causes controlled disposition of the bend in one of a straightened-out disposition and a deflected disposition.

12. The apparatus of claim 11, wherein controlled disposition by relative mutual translation of the drive tube and the core wire causes a reversible deformation of shape of the drive tube and of the core wire.

13. The apparatus of claim 7, wherein a core wire having a plurality of portions of length having a bending stiffness of different value is configured to reversibly deploy a distal initial bend having a bending stiffness of lower bending stiffness value than one of the plurality of portions of length.

14. A method for constructing a catheterization apparatus including a catheter having a steering mechanism for navigation through body vessels, the method comprising:
providing a resilient straight core wire distally that is deformed into a core wire bend forming a transition between a core wire body portion and a straight core wire nose,
providing a drive tube having a drive tube lumen,
disposing the deformed core wire in the drive tube lumen such that the core wire and the drive tube are translatable relative to each other to change a configuration thereof between: (i) a navigation configuration for navigation in bodily vessels, in which the drive tube lumen is in a straightened disposition supporting the core wire bend therein, and (ii) a penetration configuration for entering a bifurcated vessel,
wherein the core wire nose is configured to deflect a distal portion of the drive tube into a deflected drive tube arm oriented in continuation past the core wire nose, and
wherein a radiopaque marker is applied on at least one portion of length of at least one of the drive tube and the core wire to indicate a value of bending stiffness.

15. The method of claim 14, wherein the catheterization apparatus for is configured for penetration into an aortic type III arch bifurcation by performing an operation including:
navigating the drive tube with the core wire therein to a first reference location, with the drive tube distal opening extending distally away from a nose tip of the core wire,
translating the nose tip to a second reference location from where core wire is translated for erection of the drive tube arm which as result thereof, deflects away,
then translating the drive tube over the core wire and away therefrom, to create a desired length of drive tube arm,
then rotating both the drive tube and the core wire together until the drive tube arm is oriented in an appropriate angular direction aimed at an entry of the bifurcation, and
translating the drive tube along the core wire to a desired length, for engagement and support thereof in the entry of the bifurcation, and in sequence, translating the core wire out of the drive tube and into the bifurcation, whereafter the drive tube is translated over the core wire for further navigation in the bifurcation.

16. The method of claim 14, wherein the drive tube includes a distal initial bend and a plurality of portions of length having different values of bending stiffness, wherein the bending stiffness of at least one of the portions has a value superior to a bending stiffness value of the distal initial bend, and
wherein the catheterization apparatus for is configured for penetration into an aortic type III arch bifurcation by performing an operation including:
navigating a first core wire, which has a first core wire bend and is supported in the drive tube, to a first reference location, with the drive tube distal opening extending distally away from a nose tip of the first core wire,
translating the nose tip of the first core wire to a second reference location and translating the first core wire in position for erection of the drive tube arm to deflect away,
then translating the drive tube over the first core wire and away therefrom, to create a desired length of drive tube arm,
then rotating both the drive tube and the core wire together until the drive tube arm is oriented in a desired angular direction aimed at the entry of the bifurcation,
translating the drive tube along the core wire, to extend a desired length, and disposed the drive tube for engagement and support at or into an entry of the bifurcation,
retrieving the first core wire is retrieved out of the drive tube and replacing the first core wire in the drive tube with a second core wire that includes a plurality of portions of length having different values of bending stiffness, wherein the bending stiffness of at least one of the portions has a value superior to the bending stiffness value of the distal initial bend of the drive tube, and
driving the second core wire in translation into the drive tube and through the distal initial bend, for one out of the plurality of portions of length having a bending stiffness value superior to the bending stiffness value of the initial bend to deform the initial bend in straightened out disposition.

17. The method of claim 14, wherein the drive tube has a drive tube lumen via which radiopaque agents and therapeutic agents can be conveyed from a drive tube proximal opening to a drive tube distal opening and thereout.

18. The method of claim 14, wherein each one of the drive tube and the core wire is provided to include a distribution of portions of length having a bending stiffness of different value, whereby relative mutual disposition of the portions of length of the drive tube and the core wire having bending stiffnesses of different value produces a reversible controlled deformation of at least one of the drive tube and the core wire.

19. The method of claim 18, wherein each of the portions of length is one of (i) a segment of specific length having a definite bending stiffness, and (ii) a segment of specific length having a monotonously changing bending stiffness with a peak bending stiffness.

20. The method of claim 14, wherein:
the core wire has a plurality of portions of length having a bending stiffness of different value, and
the drive tube has a distal initial bend which is reversely deployable in controlled angular disposition by relative mutual translation of the drive tube and the core wire.

21. The method of claim 20, wherein the distal initial bend of the drive tube is reversibly deployable from the initial bend to a straightened-out disposition.

22. The method of claim 21, wherein the drive tube is reversibly and controllably changeable from the straightened-out disposition into a selected angular disposition.

23. A catheterization apparatus having a catheter for navigation in a lumen of a body vessel having walls, the catheter comprising:
a flexible drive tube having a smooth exterior surface supporting helically wound recessed microgrooves forming female screw threads adapted to receive therein tissue from the walls of the lumen, whereby rotation of the drive tube into protruding male screw threads formed by the tissue, which flows atraumatically to be received from over the exterior of the drive tube and into the recessed microgrooves, drives the drive tube into translation.

24. The apparatus of claim 23, wherein:
the drive tube includes a drive tube distal end made out of a stranded tube having an exterior surface which support a plurality of recessed grooves, and
the recessed grooves are microgrooves provided by interstices between coils of the stranded tube.

25. The apparatus of claim 23, wherein the microgrooves form a translation mechanism.

26. The apparatus of claim 23, wherein rotation of the drive tube rotates the drive tube distal end to provide traction force for translation into a bifurcated vessel.

27. A method for constructing a translation mechanism, the method comprising disposing microgrooves on an exterior surface of a drive tube such that the microgrooves are configured to engage lumen tissue when the drive tube is rotated.

28. A catheterization apparatus including a microcatheter for navigation through body vessels, the apparatus comprising:
a microcatheter including a drive tube which supports a core wire therein; and
an actuation device including a rotatable turntable which is configured to provide mechanical support and to operate motions of the microcatheter,
wherein actuation orders, delivered by a control station which is coupled in communication with the actuation device, control translation and radial rotation of the drive tube and of the core wire.

29. The apparatus of claim 28, wherein the actuation device is configured to dispense, retract, guide, and support a controlled length of the microcatheter, in response to the actuation orders received from the control station.

30. The apparatus of claim 29, wherein the control station operates the actuation device by remote control.

31. The apparatus of claim 28, wherein the actuation device supports a plurality of actuators and is configured to bidirectionally translate and rotate each one of the drive tube and the core wire, at a rate of precision of, respectively, sub-millimetric translation and sub-degree rotation.

32. The apparatus of claim 28, wherein the actuation device is further configured to provide a rigid guiding channel to mechanically support the microcatheter in buckling-free and in entanglement-free disposition.

33. The apparatus of claim 32, wherein the actuation device is configured as a rotatable turntable having a diameter of about 15 cm to 25 cm.

34. The apparatus of claim 32, wherein the guiding channel is concentric and close to a periphery of the rotatable turntable.

35. The apparatus of claim 32, wherein:
the drive tube is enclosed and is rigidly mechanically supported in the guiding channel, and
each one of the drive tube and the core wire is translatable and rotatable in the guiding channel.

36. The apparatus of claim 33, wherein the drive tube of the microcatheter is driven into translation by rotation of the turntable.

37. The apparatus of claim 36, wherein:
rotation of the turntable drives a controlled length of the drive tube in a distal direction by forces applied for distal penetration into a target vessel, and
the guiding channel is configured to mechanically support and guide therein the controlled length in buckling-free and in entanglement-free guiding channel compliant disposition.

38. The apparatus of claim 36, wherein the actuation device is packaged as a disposable throwaway assembly.

39. A method for constructing a catheterization apparatus including a catheter for navigation through body vessels, the catheter including a drive tube having a lumen supporting a core wire therein, the catheter being operative for penetrating into a bifurcating target vessel forming an angle with a main vessel, the method comprising:
providing computer data from a unit portion to a control station for transmission to an actuation device, and
providing the actuation device with actuators and with a channel for support of the catheter along a controlled portion of length of the channel, and for operation of the actuators according to data from the unit portion,
wherein the actuation device is operable for driving the catheter into a target vessel and for operating according to data received from the unit portion, including translation and rotatable rotation of the drive tube and of the core wire.

40. The method of claim 39, further comprising providing facilities supporting three-dimensional imaging facilities and three-dimensional computer programs, wherein the catheter is operated by digital computerized command and control.
